(19) United States
(12) Patent Application Publication
(10) Pub. No.: US 2025/0332564 A1
(43) Pub. Date: Oct. 30, 2025

(54) ISOTHERMAL REACTOR FOR PLASMA-CATALYSIS CHEMICAL CONVERSION

(71) Applicant: ENERGO, HELLEMES-LILLE (FR)

(72) Inventor: Vincent PIEPIORA, VERSAILLES (FR)

(73) Assignee: ENERGO, HELLEMES-LILLE (FR)

(21) Appl. No.: 18/690,273

(22) PCT Filed: Sep. 7, 2022
(86) PCT No.: PCT/IB2022/058410

§ 371 Date: Aug. 27, 2024

(30) Foreign Application Priority Data
Sep. 9, 2021 (FR) FR2109448

(51) Int. Cl. B01J 19/08; B01J 19/00

(52) U.S. Cl. CPC B01J 19/088; B01J 19/0013; B01J 2219/00076; B01J 2219/00132; B01J 2219/0809; B01J 2219/0815; B01J 2219/083; B01J 2219/0871; B01J 2219/0875; B01J 2219/0883; B01J 2219/0896

(57) ABSTRACT

The invention relates to a reactor for dielectric barrier discharge plasma catalysis, comprising a reactor housing (B) able to receive a plurality of DBD cells (C1, C2, C3 . . . ) removably mounted inside this housing.

### TECHNICAL FIELD

The present invention pertains to the field of chemical conversion, and relates more specifically to an isothermal reactor for the chemical conversion of molecular reagents in gaseous form into molecular products in gaseous or liquid form using a dielectric barrier discharge (DBD) plasma technology, which can if needed use a catalyst, enabling chemical conversion via plasma catalysis.

### PRIOR ART

Industrial gaseous chemical synthesis technologies use thermal heterogeneous catalysis. These technologies require high gas pressures and temperatures to promote catalyst activation, shift the thermodynamic equilibrium and guarantee an attractive conversion rate. The catalysis volumes involved in these technologies are significant, with throughput to catalyst volume ratios of several thousand per hour, requiring large reactors. In the case of exothermic or endothermic reactions, temperature control in these reactors is decisive, which makes controlling such a reactor more complex and increases its manufacturing costs. In addition, heterogeneous catalysis requires good gas quality, which means that a purification device is needed upstream of the reactor. Using this equipment entails high production costs.

A number of projects have been carried out to develop cold plasma chemical synthesis technologies, which eliminate the aforementioned temperature problems: among these technologies, dielectric barrier discharge (DBD), in particular combined with the presence of a heterogeneous cataylst (plasma catalysis), has proven particularly promising.

Various experimental reactors have been tested, but their ease of use has proven incompatible with industrial use.

One object of the invention is therefore in particular to propose a solution for carrying out DBD plasma catalysis on an industrial scale.

This object of the invention is achieved with a housing for a dielectric barrier discharge reactor in particular by plasma catalysis, comprising fixed support elements and a plurality of DBD cells suitable for installation on said support elements without disassembling these support elements.

These removable cells can thus be installed in the housing with minimum effort: this means they can be prepared outside the housing and then simply installed in the housing when it is to be used for chemical synthesis inside a reactor.

When maintenance is required, the cells are simply removed from the housing, making it easy to work on each of them without any complex dismantling operations.

This housing is therefore completely compatible with use in a reactor in an industrial context.

According to other optional features of the invention, considered alone or in combination:
said housing comprises two plates provided with bores able to receive said DBD cells, an inlet for reagent fluid, an outlet for product fluid, and an inlet and an outlet for heat transfer fluid, said support means comprising two plates provided with bores able to removably receive said DBD cells: this very simple design of the reactor enables the various fluids involved in the operation of the reactor to flow freely;
said inlets and outlets are arranged so as to allow said reagent/product fluids and said heat transfer fluid to flow in directions substantially and respectively parallel and perpendicular to the axes of said DBD cells: this arrangement enables an optimum heat exchange between the DBD cells and the heat transfer fluid, whether the chemical reaction is exothermic (heat transfer from the DBD cells to the heat transfer fluid) or endothermic (heat transfer from the heat transfer fluid to the DBD cells);
said housing comprises, in the heat transfer fluid circulation compartment, baffles arranged so as to be separated from said cells DBD by a distance substantially equal to that separating said DBD cells from one another: such an arrangement ensures that the velocity and pressure fields of the heat transfer fluid are substantially evenly distributed inside the reactor;
said housing comprises a reagent fluid inlet feeder, a product fluid outlet feeder, these feeders being provided with means for connection to said DBD cells, these feeders forming said means for supporting said DBD cells, means for electrically heating said DBD cells also being provided: this embodiment with electrical heating is suitable for endothermic chemical reactions, i.e. those requiring heat input;
said electrical heating means comprise heating sleeves surrounding each DBD cell;
said support means form the power supply ground for said DBD cells: in this way, the DBD cells can be connected to the electrical ground of the housing simply by placing the cells in the support means, without any electrical wiring.

The present invention also relates to a removable DBD cell for a housing in accordance with the above, comprising:
an electrically and thermally conductive tube,
a conductive element, held inside said tube by an upper plug made of insulating dielectric material,
a plasma-generating electrode electrically connected to said conductive element,
a lower plug closing the other end of said tube,
channels for circulating reagent fluid and product fluid, opening into the upper and lower parts of said cell respectively, and
at least one tubular element made of dielectric material arranged around said conductive element and said plasma-generating electrode and/or against the inner wall of said electrically and thermally conductive tube.

These features result in a DBD cell with a particularly simple design, which can easily be installed on and removed from the aforementioned housing.

According to other optional features of this reactive cell, considered alone or in combination:
this cell comprises an electrically conductive support and lower plug connected to said electrically and thermally conductive tube, capable of cooperating in a removable manner with the support means forming the power supply ground for said DBD cells;
said plasma-generating electrode has a greater diameter than that of said conductive element and is chosen from the group comprising a cylinder, a wire brush, a spring, a metallic conductive layer deposited inside said at least one tubular element made of dielectric material: such a plasma-generating electrode can therefore be produced very easily and at low cost with commonly available elements;
this cell is loaded with a catalyst arranged inside said electrically and thermally conductive tube, opposite said plasma-generating electrode, and held in place inside this tube by two portions of dielectric holding material arranged between said upper and lower plugs, said catalyst and said dielectric material having porosity or ducts allowing the reagent fluids to be treated to circulate: such an arrangement makes it very easy to hold the catalyst inside the electrically and thermally conductive tube, and to replace it.

The present invention also relates to a reactor in accordance with the above, comprising at least one housing fitted with removable DBD cells in accordance with the above: such a reactor, in which the DBD cells are installed in parallel, makes it possible to process large flows of reagent fluids, which makes it particularly suitable for use in an industrial context.

The removable DBD cells of this reactor are preferably interconnected by a common plasma-generating power supply.

The present invention also relates to the use of such a reactor for carrying out a chemical reaction chosen from the group comprising:

TABLE 1

| Method | Catalysts tested | Associated reactions |
|---|---|---|
| Conversion of | Pt/CeZr, Ni/Al₂O₃; | CO₂ + 4H₂ → CH₄ + 2H₂O |
| CO₂ | Ni/CeZr catalysts promoted by | |
| | Cu, La, Mn, Co, Y, Gd or Sr | |
| | α-Al₂O₃, CaTiO₃, ZrO₂, SiO₂, | CO₂ → CO + ½ O₂ |
| | BaTiO₃, TiO₂, MgO and CaO | |
| | La_{0.9}Sr_{0.1}FeO_{3+δ} perovskite, Mn/γ- | CO₂+ H₂ → CO + H₂O |
| | Al203, Ni-Fe alloy | |
| | Cu/ZnO catalysts supported on | xCO₂ + yH₂ → CₓH_{2y-4x+2z}O_{z} + |
| | zirconia promoted by Pd and Ga, | (2x − z)H₂O |
| | or Pd/ZnO and Pd/SiO₂ promoted | |
| | by Zn, Zr, Ce, Ga, Si, V, K, Ti, Cr | |
| | and Cs | |
| Conversion of | Pt/CeZr, Ni/Al₂O₃; | CO + 3H₂ → CH₄ + H₂O |
| CO | Ni/CeZr catalysts promoted by | |
| | Cu, La, Mn, Co, Y, Gd or Sr | |
| | Catalysts supported on zirconia, | xCO + yH₂ → CₓH_{2y-2x+2z}O_{z} + |
| | alumina, containing Cu/ZnO | (x − z)H₂O |
| | promoted by Pd and Ga, or | |
| | Pd/ZnO and Pd/SiO₂ promoted by | |
| | Zn, Zr, Ce, Ga, Si, V, K, Ti and Cr | |
| Conversion of | MgAl₂O₄; CNTs, Ni/γ-Al₂O₃, γ- | CH₄ → 2H₂ + C(s) |
| CH₄ | Al₂O₃, Pd/SiO₂, Pd/TiO₂, Pd/Al₂O₃, | |
| | Pt/γ-Al2O3, ZnO, ZnCr₂O₄, Cr₂O₃ | |
| | LaNiO₃@SiO₂ | CH₄ + CO₂ → 2H₂ + 2CO |
| | NiFe₂O₄/SiO₂ | |
| | 10% Ni/La₂O₃MgAl₂O₄-12% Cu- | |
| | 12% Ni/-Al₂O₃ | |
| | 10% Ni/Al₂O₃-MgO | |
| | 10% NiC600 | |
| | Ni/Al₂O₃ promoted by Ce, K. | |
| | Ni supported on Ce/Al promoted | |
| | by K and Co | |
| Synthesis of | Ru/MgO | N₂ + 3H₂ → 2NH₃ |
| NH₃ | Ru > Ni > Pt > Fe > supported on | |
| | Al₂O₃, Ru + Cs/MgO, Cu—Zn/MgO | |
| Decomposition NH₃ | MgTiO₃, CaTiO₃, SrTiO₃, and BaTiO₃ perovskites, Ni supported | ${NH}_{3} \to \frac{1}{2} {N}_{2} + \frac{3}{2} {H}_{2}$ |
| | on Al₂O₃ promoted by Fe or Co | |
| Decomposition | Al₂O₃, CdS—A12O3, or ZnS—Al₂O₃. | H₂S → H₂ + S₍ₗ₎ |
| of H₂S | SiO₂, Cr supported on Al₂O₃-doped | |
| | ZnS | |
| Water gas shift | Au/CeZrO₄, MOF (HKUST-1), | CO + H₂O → CO₂ + H₂ |
| reaction | Ni/CeOx, Mo/CeZr | |
| Synthesis of | Ni/CeOx | N₂ + O₂ → 2NO |
| NOₓ | α-Al₂O₃ | N₂ + 2O₂ → 2NO₂ |
| | 5% WO₃/γ-Al₂O₃ | |

### BRIEF DESCRIPTION OF THE DRAWINGS

Other features, aims and advantages of the invention will become apparent from the following description, which is purely illustrative and non-limiting, and which should be read with reference to the appended figures, in which:

FIG. 1 schematically shows, in axial section, a removable DBD cell according to one embodiment of the invention, comprising an electrically and thermally conductive tube and a single internal dielectric material on the plasma discharge zone of the electrode;

FIG. 2 schematically shows, in axial section, a removable DBD cell according to another embodiment of the invention, comprising an electrically and thermally conductive tube and two dielectric materials respectively arranged on the plasma discharge zone of the electrode and inside the tube;

FIG. 3 schematically shows, in axial section, a removable DBD cell according to yet another embodiment of the invention, comprising an electrically and thermally conductive tube and a single dielectric material inside the tube;

FIG. 4 schematically shows, in axial section, a removable DBD cell fixed, according to one embodiment of the invention, between the two plates of the housing of a reactor with circulation of heat transfer fluid;

FIG. 5 schematically shows, in axial section of the DBD cells, the two plates of the reactor housing with an empty space, a removable DBD cell being inserted and a fixed removable DBD cell, according to one embodiment of the invention;

FIG. 6 schematically shows, in axial section of the DBD cells, three removable DBD cells fixed to the plates of the reactor housing with their electrodes interconnected by the high-voltage connector network;

FIG. 7 schematically shows, in perspective and with a transparency effect, a removable DBD cell according to the invention;

FIG. 8 schematically shows in perspective a reactor housing according to the invention, the upper and lower covers of which have been removed in order to observe the arrangement of the fixing points for the removable DBD cells on the plates;

FIG. 9 schematically shows in perspective a reactor housing according to the invention, the upper and lower covers of which have been installed in order to observe one embodiment of the inlet and outlet ducts for the reagent/product and heat transfer fluids;

FIG. 10 schematically shows a vertical cross-section of a reactor assembly according to the invention in operation, taken along the axis of the inlet and outlet ducts for the reactant/product fluids, showing the variation in flow speed, and therefore the correct distribution between each DBD cell, of the reactant and product fluids through the removable DBD cells from the inlet duct for the reagent fluids to the outlet for the product fluids via the outlet duct;

FIG. 11 schematically shows a vertical cross-section similar to the one in FIG. 10, showing the variation in pressure of the reagent fluid as it flows through the removable DBD cells from the inlet duct for the reagent fluids to the outlet for the product fluid via the outlet duct;

FIG. 12 is a top view, i.e. in the direction Fr shown in FIG. 9, of the reactor housing according to the invention, the upper plate of which has been removed in order to observe the placement of internal baffles enabling the correct distribution of the heat transfer fluid between the longitudinal channel network of the removable DBD cells;

FIG. 13 is a top view of the reactor in operation, showing the variation in flow speed and therefore the correct distribution of the heat transfer fluid around removable DBD cells from the inlet duct for the heat transfer fluid to the outlet duct for the heat transfer fluid;

FIG. 14, similarly to FIG. 13, shows the variation in temperature of the heat transfer fluid around removable DBD cells from the inlet duct for the heat transfer fluid to the outlet duct for the heat transfer fluid;

FIG. 15, similarly to FIGS. 13 and 14, shows the variation in pressure of the heat transfer fluid around removable DBD cells from the inlet duct for the heat transfer fluid to the outlet duct for the heat transfer fluid;

FIG. 16 shows a cross-section of another embodiment of the reactor housing of the invention, which is suitable for endothermic chemical reactions and fitted with means for electrically heating the DBD cells;

FIG. 17 is a perspective view of the housing shown in FIG. 16;

FIG. 18 is a perspective view from another angle of the housing shown in FIGS. 16 and 17.

For reasons of clarity, identical or similar elements bear identical or similar reference numerals in all the figures.

The terms “upper” and “lower” will be used below: these terms should only be understood in relation to the orientation of the figures with respect to the top and bottom edges of the pages on which these figures are shown.

### DEFINITIONS

In the present invention, the term “DBD—dielectric barrier discharge” describes an electrical discharge created between two electrically conductive elements separated by one or more dielectric elements.

In the present invention, the term “DBD cell” describes a complete assembly of electrode, cathode, dielectric materials, electrical connector, fixing plugs, with preferably but not necessarily one or more catalyst(s) and one or more catalyst holding material(s), assembled in a removable cell which will be fixed to the plates of the reactor.

In the present invention, the term “reagent fluid” describes molecules that will undergo a chemical reaction, in particular by plasma catalysis, as they pass through the DBD cells of the reactor.

In the present invention, the term “product fluid” describes molecules resulting from the in particular plasma catalysis reaction inside the removable DBD cells.

In the present invention, the term “heat transfer fluid” describes a fluid suitable for transporting heat between two temperature sources.

In the present invention, the term “baffle” describes walls used to distribute the heat transfer fluid uniformly around the DBD cells.

In the present invention, the term “dielectric” describes an electrically insulating material that provides electrical insulation between the high-voltage network associated with the electrode and the electrically and thermally conductive tube connected to ground via the reactor. This type of electrically insulating material is also used inside the DBD cell to generate plasma by dielectric barrier discharge (DBD) by promoting the accumulation of electric charge between the electrode and ground up to the breakdown voltage and thus obtaining plasma.

In the present invention, the term “catalyst” describes a material promoting the chemical reaction of the reagent fluids.

In the present invention, the term “electrode” describes an element made of electrically conductive material inserted into the DBD cell and connected to the high-voltage network that links the DBD cells together to the source of the high-voltage generator.

In the present invention, the term “catalyst holding material” describes an element made of dielectric material inserted into the DBD cell that contains the catalyst in the plasma discharge zone whilst allowing the reagent fluid to flow through it.

Of course, the invention is described above by way of example. It is understood that a person skilled in the art is capable of creating various alternative embodiments of the invention without departing from the scope of the invention.

### DESCRIPTION OF THE EMBODIMENTS

### The Removable DBD Cells

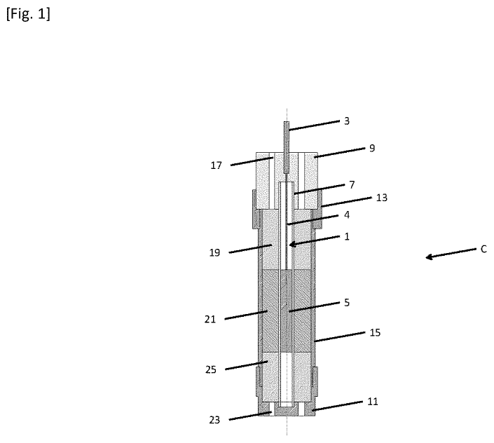

FIG. 1 schematically shows a removable DBD cell C enabling a chemical conversion to take place via a DBD plasma catalysis method with a single dielectric element. This cell comprises a conductive element 1 comprising a high-voltage connector 3 for connecting the cell to the high-voltage network of the reactor, and a conductive support 4 for connecting and holding the high-voltage connector 3 to a plasma-generating electrode 5.

The conductive element 1 marks the axial direction of the DBD cell.

The plasma-generating electrode 5, the diameter of which is greater than the conductive support 4, is used to concentrate the DBD discharges in the zone located along this electrode 5.

This plasma-generating electrode 5 can comprise metal bristles in the manner of a brush or a pipe brush. Alternatively, it can be in the form of a metal spring or metal cylinder.

The conductive element 1 is placed inside a tubular element made of dielectric material 7, against the inside wall of which the plasma-generating electrode 5 is supported, and which will enable the DBD to occur. This dielectric element 7 may be made of glass, ceramic or alumina, for example.

Alternatively, and probably more preferably, it can be provided that the dielectric material is molded over the whole of the conductive element 1 and the plasma-generating electrode 5, as in a spark plug for a combustion engine, and this dielectric material fills all the empty spaces so as to avoid the appearance of parasitic plasma phenomena.

According to one possible alternative, the plasma-generating electrode can be formed by a conductive metal layer deposited inside the tubular element made of dielectric material 7.

The conductive element 1 is held between an upper plug 9 and a lower plug 11. The upper plug 9 is made of an insulating dielectric material such as glass or ceramic and is used to hold and center the conductive element 1 (including the high-voltage connector 3). The lower plug 11 is made of a material such as a metal or metal alloy, or a ceramic. In the case of a metal alloy, which is easier to machine than a ceramic, 316L stainless steel could be used.

The upper plug 9 is fixed to an upper metallic support 13, itself fixed to an electrically and thermally conductive tube 15, defining the cylindrical outer wall of the removable DBD cell, preferably made of meal or metal alloy.

The upper plug 9 allows the reagent fluid to circulate through one or more perforated channels 17 or another porous structure allowing the reagent fluid to circulate. The upper plug 9 is also used to contain the upper portion 19 of a catalyst 21 holding material, arranged in the upper part of the tube 15.

The lower plug 11 allows the reagent fluid to circulate through one or more perforated channels 23 or another porous structure allowing the reagent fluid to circulate. The lower plug 11 is also used to contain the lower portion 25 of a catalyst holding material 21, arranged in the lower part of the tube 15.

The upper portion 19 of the catalyst 21 holding material and the lower portion 25 of the catalyst 21 holding material keep the catalyst 21 in the DBD plasma discharge zone, opposite the plasma-generating electrode 5.

The upper 19 and lower 25 portions of catalyst holding material and the catalyst 21 enable the reagent fluid to pass through thanks to their permeable nature. They are contained inside the electrically and thermall conductive tube 15 and held by the upper 9 and lower 11 plugs.

The catalyst holding material can typically comprise glass or quartz or ceramic beads, or a sintered glass material, the diameter of which will be chosen so as to prevent the catalyst grains from escaping into the inter-bead spaces. By wall of example, for catalyst grains with a diameter of 0.5 mm, beads of holding material with a diameter of 1 mm will be chosen.

The catalyst 21, located in the DBD plasma discharge zone opposite the plasma-generating electrode 5, will enable a chemical conversion of the reagent fluid to take place.

By way of example and in a non-limiting manner, the electrically and thermally conductive tube 15 can have a diameter that does not exceed the diameter of the plasma-generating electrode 5 of the conductive element 1 of 3 cm, and preferably of 2 cm. The length of this tube 15 is furthermore typically less than 30 cm.

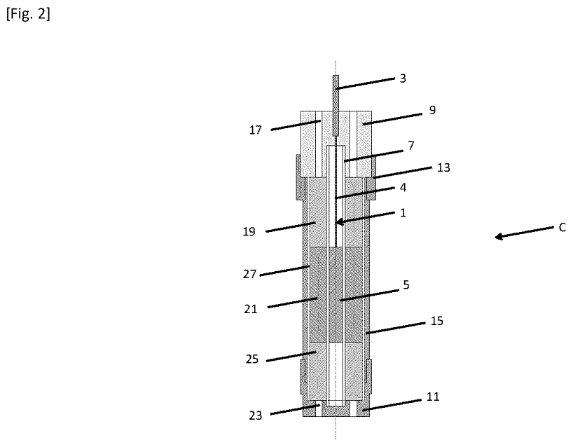

FIG. 2 shows another embodiment of a removable DBD cell according to the invention.

This embodiment differs from the previous one in that a second tubular dielectric element 27, containing the two portions 19, 25 of holding material and the catalyst 21, is arranged against the inner wall of the electrically and thermally conductive tube 15.

This embodiment therefore enables a chemical conversion to take place via a DBD plasma catalysis method with two dielectric elements 7, 27.

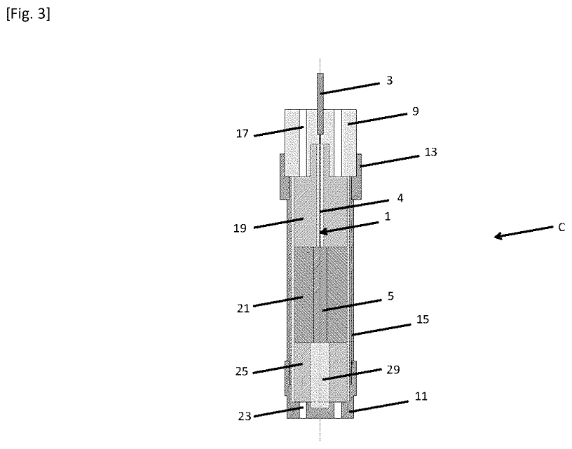

FIG. 3 shows yet another embodiment of a removable DBD cell according to the invention.

This embodiment differs from the previous one in that the conductive element 1 is no longer placed inside a tubular dielectric element 7. Only the conductive support 4 is coated with a dielectric material, and a centering element 29 made of dielectric material is interposed between the plasma-generating electrode 5 and the lower plug 11. However, in this embodiment, there is no dielectric element around the plasma-generating electrode 5.

The embodiment shown in FIG. 3, in which the plasma-generating electrode 5 is bare, reduces the breakdown voltage, promotes heat exchange between the plasma-generating electrode 5 and the outside of the catalytic cell and increases the quantity of catalyst 21 that can be integrated, all the dimensions being equal.

In the embodiments shown in FIGS. 1 to 3, there has been a catalyst 21 arranged inside the tube 15, but the invention also relates to certain applications in which DBD is to be carried out without a catalyst.

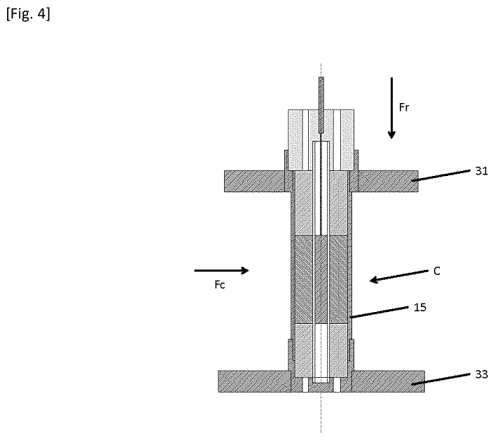

FIG. 4 schematically shows a removable DBD cell C, in accordance, for example, with the first embodiment above, which is fixed to the upper 31 and lower 33 plates of a reactor housing according to the invention.

The plates 31 and 33, which form fixed support elements, can be made of 316L grade stainless steel, for example, and are drilled in multiple places to allow a plurality of DBD cells C to be removably positioned parallel to each other. The reagent fluid flows from top to bottom, as shown by the arrow Fr in FIG. 4, through the removable DBD cell C, whilst a heat transfer fluid flows around the electrically and thermally conductive tube 15 of the DBD cell and between the two plates 31 and 33, as shown by the arrow Fc. The circulation of the heat transfer fluid enables an isothermal reactor to operate.

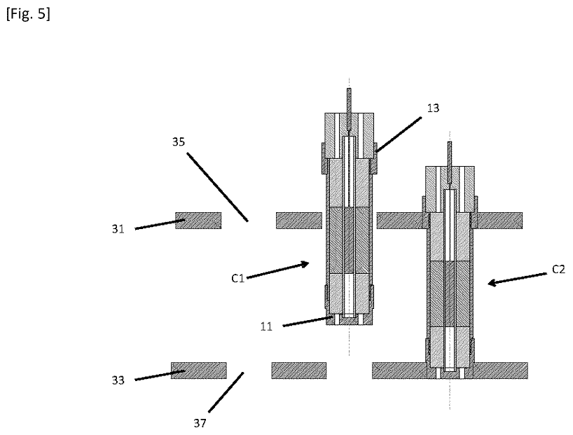

The removable DBD cells are installed on the plates 31, 33 of the housing of the reactor when the heat transfer fluid is drained. FIG. 5 schematically shows the installation of DBD cells C1, C2 between the upper 31 and lower 33 plates of the housing of the reactor. On the left-hand side of FIG. 5, there is an empty space: this space is formed by two bores 35, 37 formed respectively in the upper 31 and lower 33 plates of the housing of the reactor. In the middle of FIG. 5, a removable DBD cell C1 is being inserted into a vacant space. On the right-hand side of FIG. 5, a removable DBD cell C2 is fixed: the metallic support 13 of each cell C is held in the corresponding bore 35 of the upper plate 31, and the lower plug 11 of each cell C is held in the corresponding bore 37 of the lower plate 33.

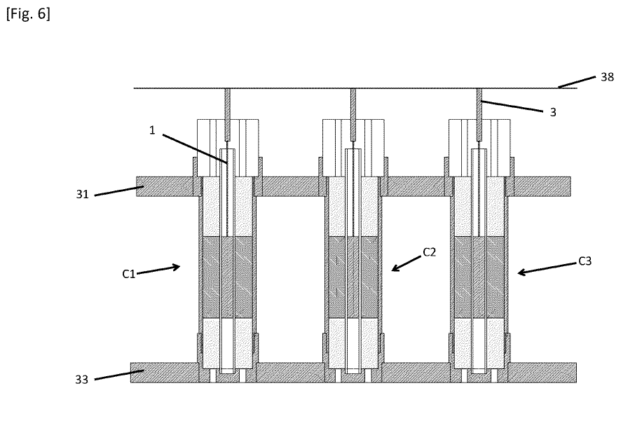

FIG. 6 schematically shows an electrical interconnection of the conductive elements 1: when all the removable DBD cells C1, C2, C3 . . . are installed between the 2 plates 31 and 33, their conductive elements 1 are connected to the high-voltage network via a network of electrical cables 38.

As can be understood from reading the above description, the removable DBD cells C can be prepared and assembled in a dedicated place, before being moved then easily installed in the housing of the DBD reactor, without disassembling the plates 31, 33. They can also be easily maintained and replaced. The electrically and thermally conductive tube 15 allows the heat flow of the isothermal reactor to be exchanged.

The conductive elements 1 of each removable DBD cell C constitute the anodes of these cells, the cathodes being made up of the electrically and thermall conductive tubes 15 of these cells, which are electrically connected to the ground of the housing B of the reactor via the upper metallic support 13, and the lower electrically and thermally conductive plug 11, themselves electrically connected to the plates 31 and 33 forming the power supply ground for the DBD cells.

### The Isothermal Plasma-Catalysis Conversion Reactor

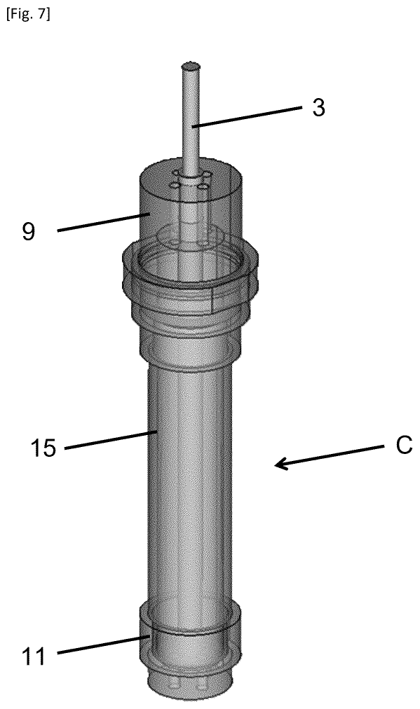

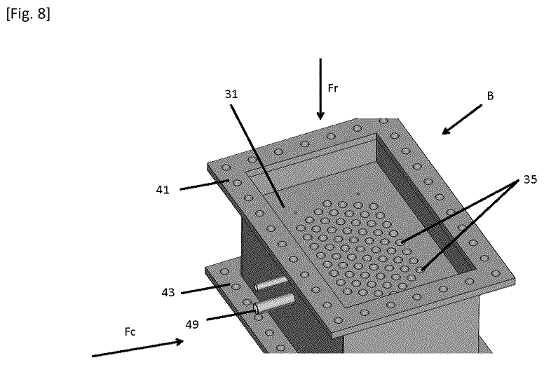

The removable DBD cells C, one of which is shown in perspective in FIG. 7, are used in an isothermal reactor housing B as shown schematically in FIG. 8. The DBD cells Care parallelized by being installed in the bores 35 of the upper plate 31, and in the corresponding bores (not shown in FIG. 8) of the lower plate of the housing B.

A high-voltage socket (not shown) connected in a sealed manner to the network of electrical cables 38 is used to supply the removable DBD cells with a signal of the desired voltage, frequency and power, as a function of the reactions that are to be carried out in the reactor.

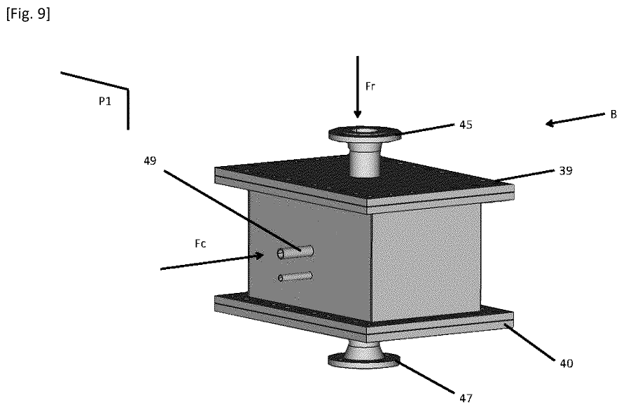

Once sealed by covers 39, 40 screwed onto upper 41 and lower 43 flanges of the housing B, a reactor is obtained enabling:
on the one hand the passage of the reagent fluid in the DBD cells arranged inside the housing, between an inlet 45 for the reagent fluid and an outlet 47 for the product fluid, in the main direction Fr shown in FIGS. 9 to 11 (according to another possible alternative, the inlet 45 and the outlet 47 could be positioned on the sides of the housing B), and
on the other hand, the passage of heat transfer fluid around the DBD cells without being in contact with the reagent fluid, in the space located between the upper 31 and lower 33 plates of the reactor housing, between an inlet 49 and an outlet 51 for heat transfer fluid, in the main direction Fc shown in FIGS. 9 and 12 to 15.

### Distribution of the Reagent Fluids

The reactor according to the invention enables the use of one or more DBD cells arranged parallel to one another and ensures that the reagent fluid is distributed evenly between the cells mainly thanks to the pressure drop induced by the passage of the reagent fluid through the various elements of the removable DBD cell. A pressure drop will make it possible to avoid a preferential path determined by the initial speed of the fluid in the inlet duct 45 due to the kinetic energy of the fluid, and instead make it possible to distribute the fluid in all the cells, the uniform flow of which in each cell will be established by the balance between flow rate and pressure drop in each cell.

In other words, the compartment of the reactor dedicated to the flow of the reagent fluid contains a network of DBD cells in which the fluid flows to the outlet of the reactor, the pressure drop in each of the cells induced by the presence of the catalyst allowing a uniform distribution of the reagent fluid in each cell placed in parallel thanks to a phenomenon of equilibrium between fluid flow rate and associated pressure drop.

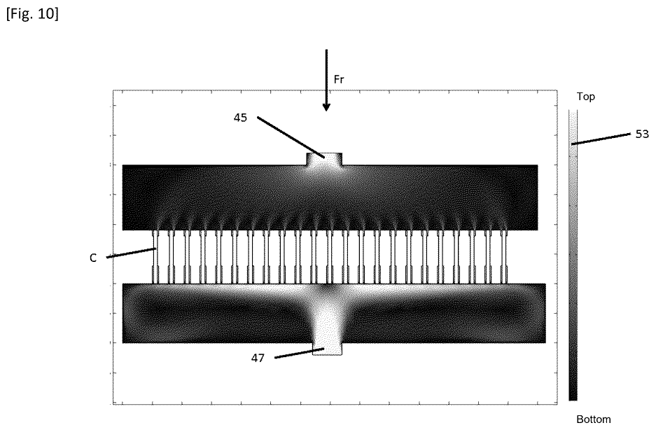

FIG. 10 schematically shows a vertical cross-section of the reactor according to the invention, i.e. taken along the plane P1 shown in FIG. 9, showing the speed 53 and the even distribution of the reagent fluid between the inlet 45 and the outlet 47 in all the DBD cells C thanks to their respective pressure drops.

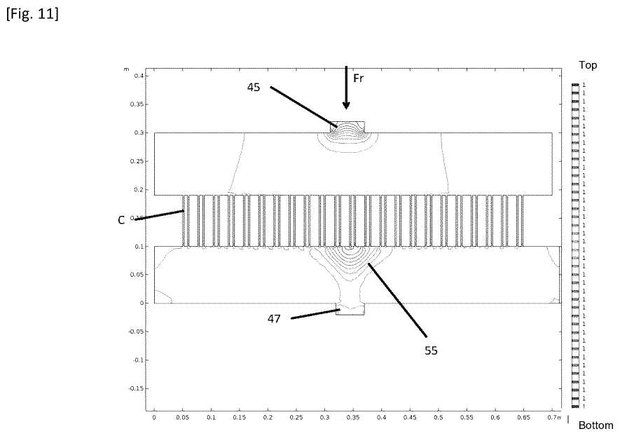

FIG. 11 shows the isobars 55 and the even distribution of the reagent fluid between the inlet 45 and the outlet 47 in all the DBD cells C thanks to their respective pressure drops.

In order to avoid resonance phenomena when using a large number of DBD cells, a non-symmetrical network of cells can be set up with a larger number of cells on one side of the reactor without affecting the even distribution of the reagent fluid.

### Distribution of the Heat Transfer Fluid

The reactor according to the invention enables the use of one or more DBD cells arranged parallel to one another.

The compartment of the reactor dedicated to the flow of heat transfer fluid contains an arrangment of walls ensuring that the heat transfer fluid is distributed evenly across all the cells; more specifically, baffles are provided to ensure that the heat transfer fluid passes through the network of parallel cells.

The baffles are ideally located at a distance from the DBD cells equivalent to the distance that separates the DBD cells from one other. The baffles also reproduce the shape of the cell network to avoid any preferential paths.

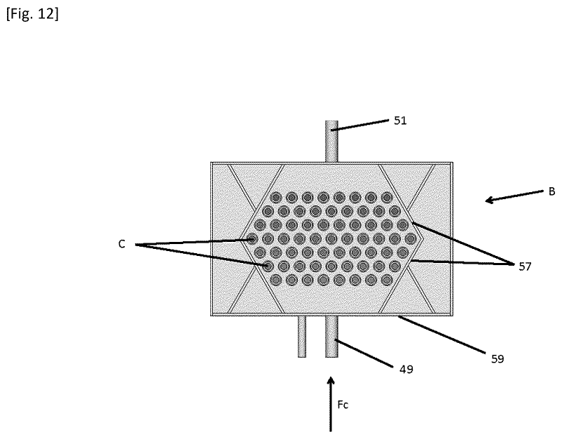

FIG. 12 schematically shows a top view of the reactor housing B with the presence of baffles 57 ideally situated along the network of DBD cells C. The heat transfer fluid flows between the inlet 49 and the outlet 51 through the network of DBD cells C and between the side walls 59 and the plates 31, 33 of the housing B.

The DBD cells C and the baffles 57 are preferably arranged so as to define a network of channels for the circulation of the heat transfer fluid having a generally hexagonal shape, as shown in FIG. 12, these channels extending in a direction substantially perpendicular to the axes of the DBD cells C.

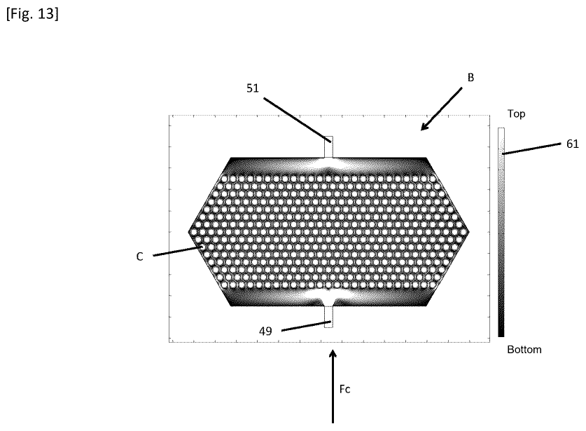

FIG. 13 shows the speed 61 of the heat transfer fluid in all the network of cells C between the inlet 49, the outlet 51 and the baffles 57: as shown in this figure, this speed is distributed substantially uniformly within the volume defined by the housing B of the reactor.

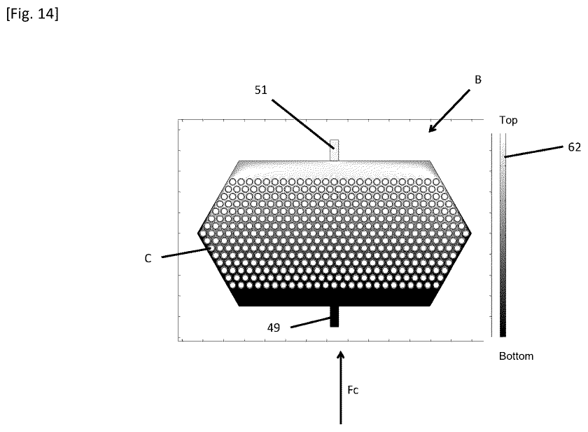

FIG. 14 shows the temperature gradient 62 of the heat transfer fluid in all the network of cells C between the inlet 49, the outlet 51 and the baffles 57.

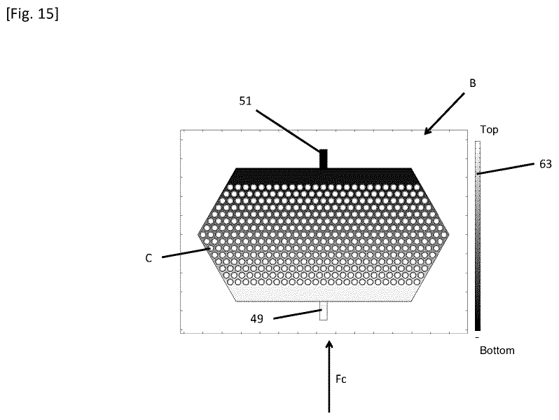

FIG. 15 shows the pressure gradient 63 of the heat transfer fluid in all the network of cells C between the inlet 49, the outlet 51 and the baffles 57.

The flow of the reagent and product fluids and of the heat transfer fluid is not influenced by the arrangement of the inlets and outlets of the reactor but rather by the arrangement of the baffles for the heat transfer fluid and by the pressure drop for the reagent and product fluids (enabling uniform distribution).

### Electrical Heating Embodiment

In the case of endothermic chemical reactions, the DBD cells need to be heated, typically to several hundred degrees celsius: in this case, the use of a heat transfer fluid such as oil is not appropriate.

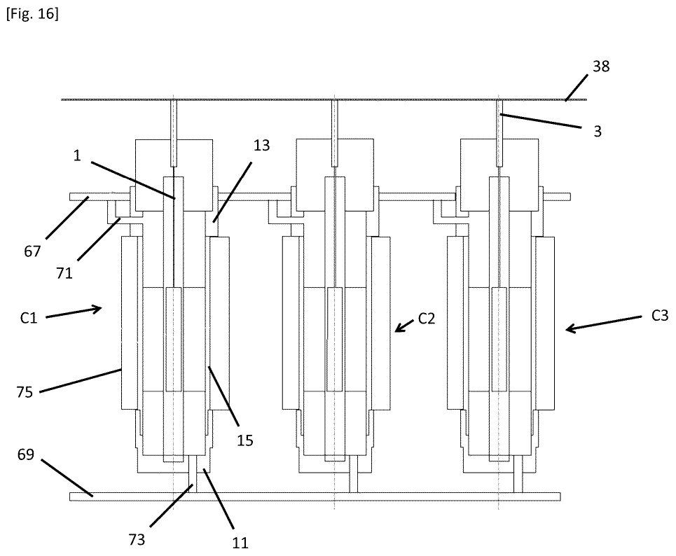

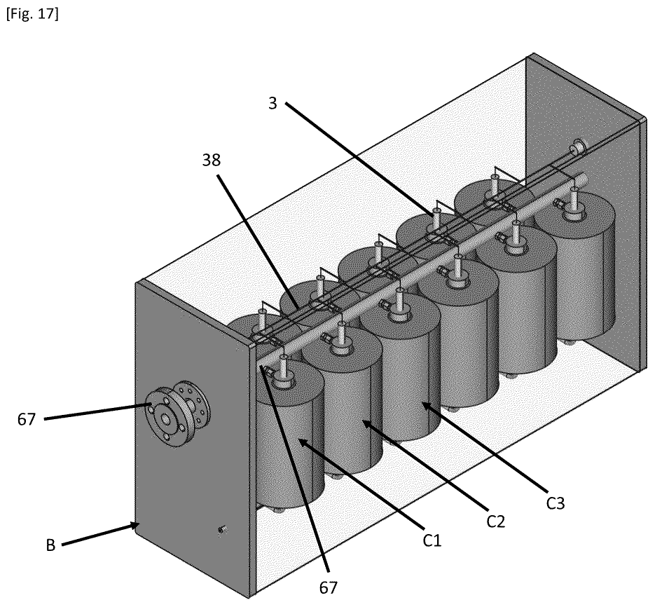

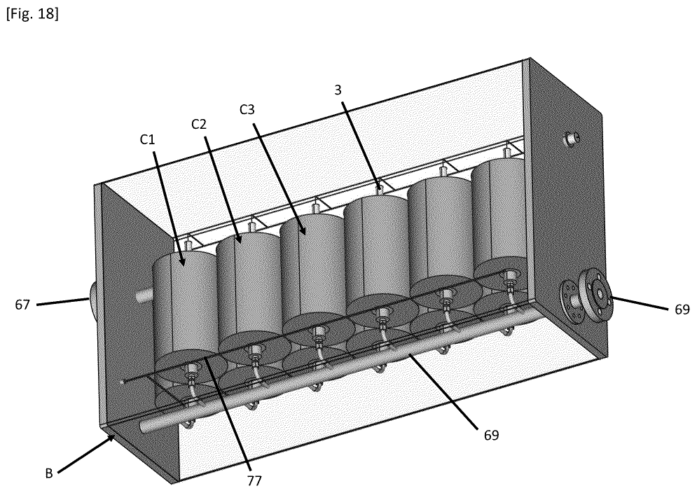

A solution for electrically heating the DBD cells is therefore conceivable, shown in the appended FIGS. 16 to 18.

As shown in these figures, the housing B comprises a reagent fluid supply feeder 67, and a product fluid outlet feeder 69, these feeders being electrically conductive.

These feeders 67, 69 are respectively connected to each of the DBD cells C1, C2, C3 . . . by electrically conductive inlet 71 and outlet 73 ducts arranged respectively in the upper support 13 and in the lower plug 11 of each cell.

Means for connection, such as connectors marketed under the Swagelok or Fitok brand names, are used to removably connect the inlet 71 and outlet 73 ducts of each DBD cell C1, C2, C3 . . . respectively to the supply 67 and outlet 69 feeders.

Thermally insulated electrical heating sleeves 75 surround each DBD cell and are powered by electrical cables 77; thermoregulators and thermocouples are used to control the temperature of these heating sleeves 75.

In this embodiment, the supply 67 and outlet 69 feeders form support elements for the DBD cells C1, C2, C3.

These electrically conductive support elements form the power supply ground for these DBD cells once they are connected to them via the electrically conductive inlet 71 and outlet 73 ducts of each cell, themselves electrically connected to the electrically and thermally conductive tube 15 of each cell.

This embodiment ensures that the reagent fluid is distributed evenly throughout the DBD cells and provides precise temperature control thanks to the individual heating sleeves and the temperature regulation by ventilated air inside the housing.

The DBD cells are very easy to install without the need to dismantle the supply and outlet feeders: a simple connection of the inlet and outlet ducts of each DBD cell to these supply and outlet feeders thanks to the connection system ensures that these DBD cells are electrically and fluidically connected to these feeders.

### EXAMPLES

The reactor with removable DBD cells that has just been described can be used very usefully and effectively for the following reactions:

TABLE 2

| Method | Catalysts tested | Associated reactions |
|---|---|---|
| Conversion of | Pt/CeZr, Ni/Al₂O₃; | CO₂ + 4H₂ → CH₄ + 2H₂O |
| CO₂ | Ni/CeZr catalysts promoted by | |
| | Cu, La, Mn, Co, Y, Gd or Sr | |
| | α-Al₂O₃, CaTiO₃, ZrO₂, SiO₂, | CO₂ → CO + ½ O₂ |
| | BaTiO₃, TiO₂, MgO and CaO | |
| | La_{0.9}Sr_{0.1}FeO_{3+δ} perovskite, Mn/γ- | CO₂ + H₂ → CO + H₂O |
| | Al2O3, Ni—Fe alloy | |
| | Cu/ZnO catalysts supported on | xCO₂ + yH₂ → CₓH_{2y-4x+2z}O_{z} + |
| | zirconia promoted by Pd and Ga, | (2x − z)H₂O |
| | or Pd/ZnO and Pd/SiO₂ promoted | |
| | by Zn, Zr, Ce, Ga, Si, V, K, Ti, Cr | |
| | and Cs | |
| Conversion of | Pt/CeZr, Ni/Al₂O₃; | CO + 3H₂ → CH₄ + H₂O |
| CO | Ni/CeZr catalysts promoted by | |
| | Cu, La, Mn, Co, Y, Gd or Sr | |
| | Catalysts supported on zirconia, | xCO + yH₂ → CₓH_{2y-2x+2z}O_{z} + |
| | alumina, containing Cu/ZnO | (x − z)H₂O |
| | promoted by Pd and Ga, or | |
| | Pd/ZnO and Pd/SiO₂ promoted by | |
| | Zn, Zr, Ce, Ga, Si, V, K, Ti and Cr | |
| Conversion of | MgAl₂O₄; CNTs, Ni/γ-Al₂O₃, γ- | CH₄ → 2H₂ + C₍ₛ₎ |
| CH₄ | Al₂O₃, Pd/SiO₂, Pd/TiO₂, Pd/Al₂O₃, | |
| | Pt/γ-Al2O3, ZnO, ZnCr₂O₄, Cr₂O₃ | |
| | LaNiO₃@SiO₂ | CH₄ + CO₂ → 2H₂ + 2CO |
| | NiFe₂O₄/SiO₂ | |
| | 10% Ni/La₂O₃MgAl₂O₄-12% Cu- | |
| | 12% Ni/-Al₂O₃ | |
| | 10% Ni/Al₂O₃-MgO | |
| | 10% NiC600 | |
| | Ni/Al₂O₃ promoted by Ce, K. | |
| | Ni supported on Ce/Al promoted | |
| | by K and Co | |
| Synthesis of | Ru/MgO | N₂ + 3H₂ → 2NH₃ |
| NH₃ | Ru > Ni > Pt > Fe > supported on | |
| | Al₂O₃, Ru + Cs/MgO, Cu—Zn/MgO | |
| Decomposition NH₃ | MgTiO₃, CaTiO₃, SrTiO₃, and BaTiO₃ perovskites, Ni supported | ${NH}_{3} \to \frac{1}{2} {N}_{2} + \frac{3}{2} {H}_{2}$ |
| | on Al₂O₃ promoted by Fe or Co | |
| Decomposition | Al₂O₃, CdS-Al2O3, or ZnS—Al₂O₃. | H₂S → H₂ + S₍ₗ₎ |
| of H₂S | SiO₂, Cr supported on Al₂O₃- | |
| | doped ZnS | |
| Water gas shift | Au/CeZrO₄, MOF (HKUST-1), | CO + H₂O → CO₂ + H₂ |
| reaction | Ni/CeOx, Mo/CeZr | |
| Synthesis of | Ni/CeOx | N₂ + O₂ → 2NO |
| NOₓ | α-Al₂O₃ | N₂ + 2O₂ → 2NO₂ |
| | 5% WO₃/γ-Al₂O₃ | |

TABLE 3

| Method | Catalysts tested | Associated reactions |
|---|---|---|
| Conversion of | Pt/CeZr, Ni/Al₂O₃; | CO₂ + 4H₂ → CH₄ + 2H₂O |
| CO₂ | Ni/CeZr catalysts promoted by | |
| | Cu, La, Mn, Co, Y, Gd or Sr | |
| | α-Al₂O₃, CaTiO₃, ZrO₂, SiO₂, | CO₂ → CO + ½ O₂ |
| | BaTiO₃, TiO₂, MgO and CaO | |
| | La_{0.9}Sr_{0.1}FeO_{3+δ} perovskite, Mn/γ- | CO₂ + H₂ → CO + H₂O |
| | Al2O3, Ni—Fe alloy | |
| | Cu/ZnO catalysts supported on | xCO₂ + yH₂ → CₓH_{2y-4x+2z}O_{z} + |
| | zirconia promoted by Pd and Ga, | (2x − z)H₂O |
| | or Pd/ZnO and Pd/SiO2 promoted | |
| | by Zn, Zr, Ce, Ga, Si, V, K, Ti, Cr | |
| | and Cs | |
| Conversion of | Pt/CeZr, Ni/Al₂O₃; | CO + 3H₂ → CH₄ + H₂O |
| CO | Ni/CeZr catalysts promoted by | |
| | Cu, La, Mn, Co, Y, Gd or Sr | |
| | Catalysts supported on zirconia, | xCO + yH₂ → CₓH_{2y-2x+2z}O_{z} + |
| | alumina, containing Cu/ZnO | (x − z)H₂O |
| | promoted by Pd and Ga, or | |
| | Pd/ZnO and Pd/SiO₂ promoted by | |
| | Zn, Zr, Ce, Ga, Si, V, K, Ti and Cr | |
| Conversion of | MgAl₂O₄; CNTs, Ni/γ-Al₂O₃, γ- | CH₄ → 2H₂ + C₍ₛ₎ |
| CH₄ | Al₂O₃, Pd/SiO₂, Pd/TiO₂, Pd/Al₂O₃, | |
| | Pt/γ-Al2O3, ZnO, ZnCr₂O₄, Cr₂O₃ | |
| | LaNiO₃@SiO₂ | CH₄ + CO₂ → 2H₂ + 2CO |
| | NiFe₂O₄/SiO₂ | |
| | 10% Ni/La₂0₃MgAl₂O₄-12% Cu- | |
| | 12% Ni/-Al₂O₃ | |
| | 10% Ni/Al₂O₃-MgO | |
| | 10% NiC600 | |
| | Ni/Al₂O₃ promoted by Ce, K. | |
| | Ni supported on Ce/Al promoted | |
| | by K and Co | |
| Synthesis of | Ru/MgO | N₂ + 3H₂ → 2NH₃ |
| NH₃ | Ru > Ni > Pt > Fe > supported on | |
| | Al₂O₃, Ru + Cs/MgO, Cu—Zn/MgO | |
| Decomposition NH₃ | MgTiO₃, CaTiO₃, SrTiO₃, and BaTiO₃ perovskites, Ni supported | ${NH}_{3} \to \frac{1}{2} {N}_{2} + \frac{3}{2} {H}_{2}$ |
| | on Al₂O₃ promoted by Fe or Co | |
| Decomposition | Al₂O₃, CdS-Al2O3, or ZnS-Al₂O₃. | H₂S → H₂ + S₍ₗ₎ |
| of H₂S | SiO₂, Cr supported on Al₂O3- | |
| | doped ZnS | |
| Water gas shift | Au/CeZrO₄, MOF (HKUST-1), | CO + H₂O → CO₂ + H₂ |
| reaction | Ni/CeOx, Mo/CeZr | |
| Synthesis of | Ni/CeOx | N₂ + O₂ → 2NO |
| NOₓ | α-Al₂O₃ | N₂ + 2O₂ → 2NO₂ |
| | 5% WO₃/γ-Al₂O₃ | |

1. Housing for a dielectric barrier discharge (DBD) reactor in particular by plasma catalysis, comprising fixed support elements (31, 33; 67, 69) and a plurality of DBD cells (C1, C2, C3 . . . ) suitable for installation on said support elements (31, 33; 67, 69) without disassembling these support elements.

2. The housing (B) of claim 1, comprising an inlet (45) for reagent fluid, an outlet (47) for product fluid, and an inlet (49) and an outlet (51) for heat transfer fluid, said support means comprising two plates (31, 33) provided with bores (35, 37) able to removably receive said DBD cells (C1, C2, C3 . . . ).

3. The housing (B) of claim 2, wherein said inlets (45, 49) and outlets (47, 51) are arranged to allow said reagent/product fluids and said heat transfer fluid to flow in directions (Fr, Fc) substantially and respectively parallel and perpendicular to the axes of said DBD cells (C1, C2, C3 . . . ).

4. The housing (B) of claim 2, wherein said housing (B) comprises, in the heat transfer fluid circulation compartment, baffles (57) arranged so as to be separated from said cells DBD (C1, C2, C3 . . . ) by a distance substantially equal to that separating said DBD cells (C1, C2, C3 . . . ) from one another.

5. The housing (B) of claim 1, comprising a reagent fluid inlet feeder (67), a product fluid outlet feeder (69), these feeders being provided with means for connection to said DBD cells (C1, C2, C3 . . . ), these feeders (67, 69) forming the means for supporting said DBD cells, means (75) for electrically heating said DBD cells also being provided.

6. The housing (B) of claim 5, wherein said electrical heating means comprise heating sleeves (75) surrounding each DBD cell (C1, C2, C3 . . . ).

7. The housing (B) of claim 2, wherein said support means (31, 33; 67, 69) form the electrical power supply ground for said DBD cells (C1, C2, C3 . . . ).

8. Removable DBD cell (C) for the housing of claim 2, comprising:
an electrically and thermally conductive tube (15),
a conductive element (1), held inside said tube (15) by an upper plug (9) made of insulating dielectric material,
a plasma-generating electrode (5) electrically connected to said conductive element (1),
a lower plug (11) closing the other end of said tube (15),
channels (17, 23) for circulating reagent fluid and product fluid, opening into the upper and lower parts of said cell respectively, and
at least one tubular element made of dielectric material (7, 27) arranged around said conductive element (1) and said plasma-generating electrode (5) and/or against the inner wall of said electrically and thermally conductive tube (15).

9. The DBD cell (C) of claim 8, comprising an electrically conductive support (13) and lower plug (11) connected to said electrically and thermally conductive tube (15), capable of cooperating in a removable manner with the support means (31, 33; 67, 69) forming the electrical power supply ground for said DBD cells (C1, C2, C3 . . . ).

8. The DBD cell (C) of claim 8, wherein said plasma-generating electrode (5) has a greater diameter than that of said conductive element (1) and is chosen from the group comprising a cylinder, a wire brush, a spring, a metallic conductive layer deposited inside said at least one tubular element made of dielectric material (7).

11. The DBD cell (C) of claim 8, loaded with a catalyst (21) arranged inside said electrically and thermally conductive tube (15), opposite said plasma-generating electrode (5), and held in place inside this tube by two portions of dielectric holding material (19, 25) arranged between said upper (9) and lower (11) plugs, said catalyst (21) and said dielectric material having porosity or ducts allowing the reagent fluids to be treated to circulate.

12. Reactor comprising at least the housing (B) of claim 2 fitted with removable DBD cells (C1, C2, C3 . . . ) comprising:
an electrically and thermally conductive tube (15),
a conductive element (1), held inside said tube (15) by an upper plug (9) made of insulating dielectric material,
a plasma-generating electrode (5) electrically connected to said conductive element (1),
a lower plug (11) closing the other end of said tube (15),
channels (17, 23) for circulating reagent fluid and product fluid, opening into the upper and lower parts of said cell respectively, and
at least one tubular element made of dielectric material (7, 27) arranged around said conductive element (1) and said plasma-generating electrode (5) and/or against the inner wall of said electrically and thermally conductive tube (15).

13. The reactor of claim 12, wherein the removable DBD cells (C1, C2, C3 . . . ) are interconnected by a plasma-generating electrical power supply (38).

14. Method of using the reactor of claim 12 for carrying out a chemical reaction chosen from the group comprising:
TABLE 3

| Method | Catalysts tested | Associated reactions |
|---|---|---|
| Conversion of | Pt/CeZr, Ni/Al₂O₃; | CO₂ + 4H₂ → CH₄ + 2H₂O |
| CO₂ | Ni/CeZr catalysts promoted by | |
| | Cu, La, Mn, Co, Y, Gd or Sr | |
| | α-Al₂O₃, CaTiO₃, ZrO₂, SiO₂, | CO₂ → CO + 1/2 O₂ |
| | BaTiO₃, TiO₂, MgO and CaO | |
| | La_{0.9} Sr_{0.1}FeO_{3+δ} perovskite, Mn/γ- | CO₂ + H₂ → CO + H₂O |
| | Al2O3, Ni—Fe alloy | |
| | Cu/ZnO catalysts supported on | xCO₂ + yH₂ → CₓH_{2y-4x+2z}O_{z} + |
| | zirconia promoted by Pd and Ga, | (2x − z)H₂O |
| | or Pd/ZnO and Pd/SiO₂ promoted | |
| | by Zn, Zr, Ce, Ga, Si, V, K, Ti, Cr | |
| | and Cs | |
| Conversion of | Pt/CeZr, Ni/Al₂O₃; | CO + 3H₂ → CH₄ + H₂O |
| CO | Ni/CeZr catalysts promoted by | |
| | Cu, La, Mn, Co, Y, Gd or Sr | |
| | Catalysts supported on zirconia, | xCO + yH₂ → CₓH_{2y-2x+2z}O_{z} + |
| | alumina, containing Cu/ZnO | (x − z)H₂O |
| | promoted by Pd and Ga, or | |
| | Pd/ZnO and Pd/SiO₂ promoted by | |
| | Zn, Zr, Ce, Ga, Si, V, K, Ti and | |
| | Cr | |
| Conversion of | MgAl₂O₄; CNTs, Ni/γ-Al₂O₃, γ- | CH₄ → 2H₂ + C₍ₛ₎ |
| CH₄ | Al₂O₃, Pd/SiO₂, Pd/TiO₂, | |
| | Pd/Al₂O₃, Pt/γ-Al2O3, ZnO, | |
| | ZnCr₂O₄, Cr₂O₃ | |
| | LaNiO₃@SiO₂ | CH₄+ CO₂ → 2H₂ + 2CO |
| | NiFe₂O₄/SiO₂ | |
| | 10% Ni/La₂O₃MgAl₂O₄-12% Cu- | |
| | 12% Ni/-Al₂O₃ | |
| | 10% Ni/Al₂O₃-MgO | |
| | 10% NiC600 | |
| | Ni/Al₂O₃ promoted by Ce, K. | |
| | Ni supported on Ce/Al promoted | |
| | by K and Co | |
| Synthesis of | Ru/MgO | N₂ + 3H₂ → 2NH₃ |
| NH₃ | Ru > Ni > Pt > Fe > supported on | |
| | Al₂O₃, Ru + Cs/MgO, Cu—Zn/ | |
| | MgO | |
| Decomposition NH₃ | MgTiO₃, CaTiO₃, SrTiO₃, and BaTiO₃ perovskites, Ni supported | ${NH}_{3} \to \frac{1}{2} {N}_{2} + \frac{3}{2} {H}_{2}$ |
| | on Al₂O₃ promoted by Fe or Co | |
| Decomposition | Al₂O₃, CdS-Al2O3, or ZnS— | H₂S → H₂ + S₍ₗ₎ |
| of H₂S | Al₂O₃. SiO₂, Cr supported on | |
| | Al₂O₃ - doped ZnS | |
| Water gas shift | Au/CeZrO₄, MOF (HKUST-1), | CO+ H₂O → CO₂ + H₂ |
| reaction | Ni/CeOx, Mo/CeZr | |
| Synthesis of | Ni/CeOx | N₂ + O₂ → 2NO |
| NOₓ | α-Al₂O₃ | N₂ + 2O₂ → 2NO₂ |
| | 5% WO₃/γ-Al₂O₃ | |
